(19) United States
(12) Patent Application Publication
(10) Pub. No.: US 2025/0336488 A1
(43) Pub. Date: Oct. 30, 2025

(54) BLOCKCHAIN-BASED BIOLOGICAL SAMPLE STORAGE

(71) Applicant: ENZO BIOCHEM, INC., Farmingdale, NY (US)

(72) Inventors: Hamid ERFANIAN, Farmingdale, NY (US); Adel PERALTA, Farmingdale, NY (US); Rich PALADINO, Farmingdale, NY (US)

(73) Assignee: ENZO BIOCHEM, INC., Farmingdale, NY (US)

(21) Appl. No.: 18/865,197

(22) PCT Filed: May 11, 2023
(86) PCT No.: PCT/US2023/021816

§ 371 Date: Nov. 12, 2024

Related U.S. Application Data
(60) Provisional application No. 63/341,134, filed on May 12, 2022.

(51) Int. Cl. G16H 10/40; G16H 10/60

(52) U.S. Cl. CPC G16H 10/40; G16H 10/60

(57) ABSTRACT

A computer-implemented system, method, and computer program product for managing the storage and handling of biological samples and related information using a blockchain stored on a network of computing nodes.

### BACKGROUND

The present application relates generally to information handling and/or data processing networks, and more particularly to providing user's access (e.g., remote access) to biological samples or specimens stored on a computing network using a blockchain process.

Biological specimens (e.g., biospecimens, materials or samples) have been collected and stored from individuals in both clinical and research settings. Such specimens or samples can include blood, sputum, serum, urine, bodily tissues, etc.

Typically, on the clinical side, a subject's biological specimens (e.g., blood samples) are obtained at a doctor's office, a hospital or an authorized lab as ordered by a doctor or other medical professional. The biological specimen is then subject to processing and clinical analysis and the subject's diagnosis or treatment is provided and stored in an electronic medical record. After the clinical use/analysis and diagnostics are obtained for the subject's sample, any excess sample materials are typically discarded.

With respect to research setting, biological specimens, e.g., blood samples, are typically obtained with informed consent of the patient prior to the specimen draw and remain under the control of an investigator's or researcher's laboratory. Such samples may also be stored at medical institutions, (e.g., university) hospital laboratories or dedicated storage facilities. Any access to such stored samples and/or information about the sample(s) by other third-party researchers or investigators who may want to conduct research, are performed individually and in an ad hoc manner, e.g., requiring the researchers to directly query the storage facilities, the doctors' office, the laboratories, etc. for such usage/request of the sample and/or related information. Depending upon the nature of the samples/information sought by researchers, accessibility to such samples and related data can be arduous, requiring multiple inquiries to multiple different medical institutions at many different geographic locations.

Currently, there is no comprehensive mechanism for third-party investigators/researchers to access physical specimens/sample or access comprehensive information about the stored biological specimens/samples. That is, third-party researchers who want to use any stored/remaining biospecimens/samples or portions thereof to perform research unrelated to the original clinical use or research study, or who want information about the samples for a research study, have no comprehensive mechanism to locate and/or access the biospecimens/samples themselves and/or obtain, manage any information relating to the samples.

### SUMMARY

The summary of the disclosure is given to aid understanding of, and not with an intent to limit the disclosure. The present disclosure is directed to a person of ordinary skill in the art. It should be understood that various aspects and features of the disclosure may advantageously be used separately in some circumstances or instances, or in combination with other aspects, embodiments, and/or features of the disclosure in other circumstances or instances. Accordingly, variations and modifications may be made to the system and/or method to achieve different effects. In this regard it will be appreciated that the disclosure presents and describes one or more inventions, and in aspects includes numerous inventions as defined by the claims.

A system, method and/or computer program product is disclosed for managing the storage and handling of biological samples and related information using a blockchain stored on a network of computing nodes.

The system, method and/or computer program product stores information about biospecimens/samples including the geographic location of the facility where such biospecimens/samples are stored and disseminates such information to different entities.

In an embodiment, a blockchain-based system, method and/or computer program product is provided for storing biospecimens/samples and related information and enabling access to the stored samples.

In one aspect, there is provided a computer-implemented method for managing the storing and handling of biological samples using a network of computing nodes. The method comprises: receiving, at a processor of a computing node, a message request to store information relating to a biological sample taken from a subject; extracting from the message request, using the processor, location information associated with the subject's stored physical biological sample; obtaining, using the processor, data associated with a subject providing the stored sample; appending, using the processor, a transaction to a blockchain stored on one or more computing nodes of the network, the transaction comprising information about the subject's biological sample stored, the associated location information of the subject's stored sample and the data associated with the subject; receiving, using said processor, queries pertaining to stored biological samples; and responding to said queries, using the processor, by accessing the information within one or more transactions appended to said blockchain.

A non-transitory computer readable medium that includes programming instructions is also disclosed that, when executed by at least one hardware processor, configure the at least one hardware processor to perform the processes and/or steps discussed above.

The foregoing and other objects, features, and/or advantages of the invention will be apparent from the following more particular descriptions and exemplary embodiments of the invention as illustrated in the accompanying drawings wherein like reference numbers generally represent like parts of the illustrative embodiments of the invention.

### BRIEF DESCRIPTION OF THE DRAWINGS

The various aspects, features, and/or embodiments of a blockchain-based system, method and/or computer program product for managing the storage and handling of biospecimens/samples and related information and enabling access to the stored samples/information will be better understood when read in conjunction with the figures provided. Embodiments are provided in the figures for the purpose of illustrating aspects, features, and/or various details of the systems and methods, but the claims should not be limited to the precise arrangement, features, aspects, embodiments, systems, modules, functional units, programming, instructions, methods, processes, techniques, and/or devices shown, and the arrangements, features, aspects, embodiments, systems, modules, functional units, programming, instructions, methods, processes, techniques, and/or devices shown may be used singularly or in combination with other arrangements, features, aspects, embodiments, systems, modules, functional units, programming, instructions, methods, techniques, processes, and/or devices.

FIG. 1 depicts a plurality of peer devices in communication over a communications network that form a blockchain network;

FIG. 2 is a block diagram of a computer system used in accordance with one or more embodiments of the present disclosure;

FIG. 3 is a system diagram illustrating a blockchain in accordance with an aspect of the present disclosure;

FIGS. 4A-4B depict both a blockchain approval and blockchain denial workflow methodology according to aspects of the present disclosure;

FIG. 5 illustrates a diagrammatic flowchart of a method to store a biological sample and related information to the blockchain node according to an embodiment of the present disclosure;

FIG. 6 illustrates a diagrammatic flowchart of a method for processing queries received at the blockchain for obtaining a physical sample and/or queries requesting information relating to samples stored on the blockchain according to an embodiment of the present disclosure; and

FIG. 7 illustrates a diagrammatic flowchart of a method implemented at blockchain cleanse engine to anonymize patient data related to that patient's sample to be stored on the blockchain according to an embodiment of the present disclosure.

### DETAILED DESCRIPTION

The following description is made for illustrating the general principles of the invention and is not meant to limit the inventive concepts claimed herein. In the following detailed description, numerous details are set forth in order to provide an understanding of the system, method, and/or techniques of the invention for securely storing a biospecimen/sample on a blockchain and facilitate the management of samples including the accessing of samples and obtaining of information related to samples, e.g., for research purposes. It will be understood, however, by those skilled in the art that different and numerous embodiments of the system and/or method of the invention may be practiced without the specific details, and the claims and disclosure should not be limited to the arrangements, systems, devices, modules, functional units, programming, instructions, embodiments, features, aspects, processes, methods, techniques, and/or details specifically described and shown herein. Further, particular features, aspects, embodiments, arrangements, systems, devices, modules, functional units, programming, instructions, methods, processes, techniques, details, etc. described herein can be used in combination with other described features, aspects, embodiments, arrangements, structures, systems, devices, modules, functional units, programming, instructions, techniques, methods, processes, details, etc. in each of the various possible combinations and permutations.

It is assumed that those skilled in the art are familiar with computing environments, including networked computing environments, including for example obtaining secure access to a network of computing nodes. It is further assumed that those skilled in the art are familiar with blockchain technology. Blockchain technology had been developed as a way of providing a publicly transparent and decentralized ledger that is configured to track and store digital transactions in a publicly verifiable, secure, and hardened manner to prevent tampering or revision.

The following discussion omits or only briefly describes conventional features of computing networks, including computing network architecture, blockchain technology, remote accessing into a computer node, and their operations, which should be apparent to those skilled in the art. It may be noted that a numbered element is numbered according to the figure in which the element is introduced and is typically referred to by that number throughout succeeding figures.

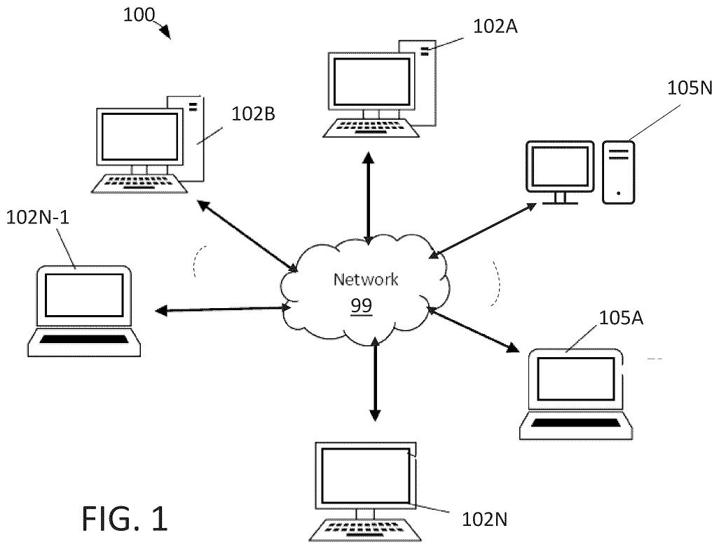

FIG. 1 illustrates an embodiment of a blockchain computing network 100 for storing biological samples and accessing related information about the biological samples stored. The network 100 includes multiple peer devices 102A, 102B, . . . , 102N, referred to as computing nodes, communicating via communications protocols across a physical communications network 99. The peer devices 102A, 102B, . . . , 102N can take many forms, for example, a computer, laptops, smart phones, personal assistants, terminals, a virtual computer, or virtual machine (VM), or a computing node. In the computing network 100, any of the peer devices 102A, 102B, . . . , 102N can serve as a host or a client of the other peer devices and one or more may host the whole or portion of a blockchain. When hosting a blockchain, peer computing device 102A, 102B, . . . etc. is referred to as a blockchain node that includes a memory for storing at least a portion of a blockchain or blockchain ledger.

Further, in FIG. 1, network 100 is depicted as including computing devices 105A, . . . , 105N representing client computer devices associated with authorized users who have credentials or permissions to access the blockchain network 100, e.g., either through a web-based portal or a distributed client application at the authorized user's device. In embodiments, the peer devices 102A, 102B, . . . 102N include application software that provides an interface enabling the users to interact with the blockchain through a distributed client application “front-end” at the authorized user's device, e.g., client device 105A, . . . , 105N. The “back-end” business logic for interacting with the blockchain is in the form of one or more smart contracts that communicate with the underlying technology of the blockchain at a node. The smart contract essentially functions as application program interface (API) connectors for connecting the distributed client application to the blockchain.

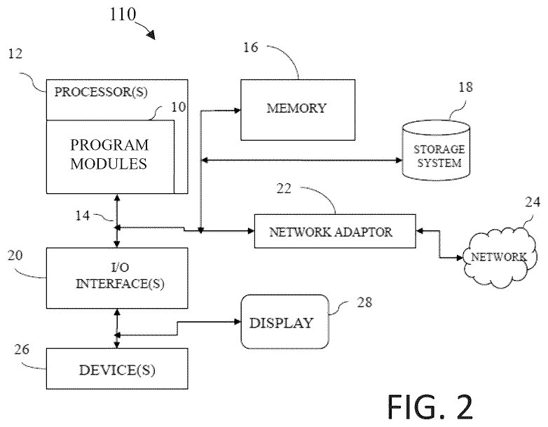

FIG. 2 illustrates an example computing system 110 in accordance with the present disclosure that can be used as a peer device 102A, . . . ,102N or client device 105A, . . . , 105N in a computing network. Hereinafter, reference to 110 is a generic term for a computer node or a computer system of which peer devices 102A, 102B, . . . 102N and client device 105A, . . . , 105N are examples. As used herein, the terms computer system and computing node are synonymous and are used interchangeably. It is to be understood that the computer system or computing node 110 depicted is only one example of a suitable electronic computer system and is not intended to suggest any limitation as to the scope of use or functionality of embodiments of the present invention. For example, the system shown may be operational with numerous other general-purpose or special-purpose computing system environments or configurations. Examples of well-known computing systems, environments, and/or configurations that may be suitable for use with the system shown in FIG. 2 may include, but are not limited to, mainframe computer systems, server computer systems, thin clients, thick clients, personal computers, networked computers, minicomputer systems, handheld or laptop devices, tablets, smart phones, multiprocessor systems, microprocessor-based systems, set top boxes, programmable consumer electronics, and distributed cloud computing environments that include any of the above systems or devices, and the like.

In some embodiments, the computer system or computing node 110 may be described in the general context of computer system executable instructions, embodied as program modules or software programs stored in memory 16, being executed by the computer system. Generally, program modules may include routines, programs, objects, components, logic, data structures, and so on that perform particular tasks and/or implement particular input data and/or data types in accordance with the present invention.

The components of the computer system 110 may include, but are not limited to, one or more processors or processing units 12, a memory 16, and a bus 14 that operably couples various system components, including memory 16 to processor 12. In some embodiments, the processor 12 may execute one or more program modules 10 that are loaded from memory 16, where the program module(s) embody software (program instructions) that cause the processor to perform one or more method embodiments pertaining to access, use and processing at the blockchain. In some embodiments, program module 10, e.g., software programs, may be programmed into the circuits of the processor 12, loaded from memory 16, storage system 18, network 24, and/or combinations thereof. It is generally appreciated that processor 12 contains circuits including integrated circuits to perform operations of the processor 12.

Bus 14 may represent one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

The computer system 110 may include a variety of computer system readable media. Such media may be any available media that is accessible by the computer system, and it may include both volatile and non-volatile media, removable and non-removable media. Memory 16 (sometimes referred to as system memory) can include computer readable media in the form of volatile memory, such as random-access memory (RAM), cache memory, and/or other forms of memory. Computer system 110 can further include other removable/non-removable, volatile/non-volatile computer system storage media. By way of example only, storage system 18 can be provided for reading from and writing to a non-removable, non-volatile magnetic media (e.g., a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 14 by one or more data media interfaces.

The computer system 110 can also communicate with one or more external devices 26 such as a keyboard, a pointing device, a display 28, etc.; one or more devices that enable a user to interact with the computer system; and/or any devices (e.g., network card, modem, etc.) that enable the computer system to communicate with one or more other computing devices. Such communication can occur via Input/Output (I/O) interfaces 20.

Computer system or computing node 110 can communicate with one or more networks 24 such as a local area network (LAN), a general wide area network (WAN), a private network, a public network (e.g., the Internet) and/or a closed network via network adapter 22. In the present disclosure, one or more of the networks 24 with which computer system 110 would communicate would connect computing node 110 to one or more other nodes in a network, and in an embodiment a closed network. As depicted, network adapter 22 communicates with the other components of computer system via bus 14. It should be understood that although not shown, other hardware and/or software components could be used in conjunction with the computer system. Examples include, but are not limited to: microcode, device drivers, redundant processing units, external disk-drive arrays, RAID systems, tape drives, and data archival storage systems, etc.

Referring to FIG. 1, one or more peer devices 102A-102N of network 100 can run/store the blockchain in a memory. The blockchain may be a public blockchain that is permissionless and enables any user to pseudo-anonymously join; or it may be a private (managed) blockchain that restricts access to the blockchain network and requires permissions; or it may be a hybrid blockchain such as a consortium or federated blockchain (having public and private elements). Typically, any blockchain functions as a database and provides three primary functions: read, write, and validate. For example, a user of the blockchain is provided with the ability to read the data that resides on the blockchain. A user of the blockchain also has the ability to write, e.g., append, data to the blockchain. Every write operation starts out as a proposed transaction that is posted on the network 100. The proposed transaction may not always be valid, for example, it may be malformed (syntax errors), or it may constitute an attempt to perform a task for which the submitter is not authorized. Validation refers to filtering out invalid transactions and then deciding on the exact order for the remaining, valid, transactions to be appended to the blockchain. This process is often called consensus.

With reference to FIG. 1, in some aspects, every computing node 110 may store the entire blockchain or a portion thereof. In some aspects, some or all of nodes 110 may also be validator nodes which, in one embodiment, perform a series of mathematical computations to determine whether a transaction is valid and also determines whether the transaction complies with the rules of the blockchain. Thus, each time a transaction is made, it is broadcasted to the entire network of validator nodes. Upon hearing the broadcasts, miners take a bunch of transactions, validate that they are “legitimate”—and put them into a block. A consensus of a set of validator nodes is required to add the transaction to a block for appending to the network 100. That is, once a block winner is picked, the blockchain validators verify that the block is valid. If a consensus is reached, then the block is added to the blockchain. Validator nodes can implement a Proof of Stake (PoS) algorithm utilizing randomly selected miners to validate transactions; or a Proof of Work (PoW) validation method. However, other blockchain transaction validation methods can be applied as known in the art, e.g., pBFT (Practical Byzantine Fault Tolerance), Kafka and Raft consensus algorithms.

Once a chronologically exact order of valid transactions to be appended to the blockchain is determined, the transactions are packaged into blocks which are in turn appended to the blockchain. Each new block that is appended to the blockchain also includes a hash of the previous block. Accordingly, as each new block is added, the security and integrity of the entire blockchain is further enhanced as each block is cryptographically linked to its immediate prior block on the blockchain. It is important to note that once data is written to the blockchain, for example, once a block including transactions has been appended to the blockchain, that data can no longer be altered or modified. In a typical blockchain, the anonymity of the users is protected, e.g., through the use of pseudonyms, and the transaction data itself is protected through the use of cryptography, e.g., via the use of hash codes.

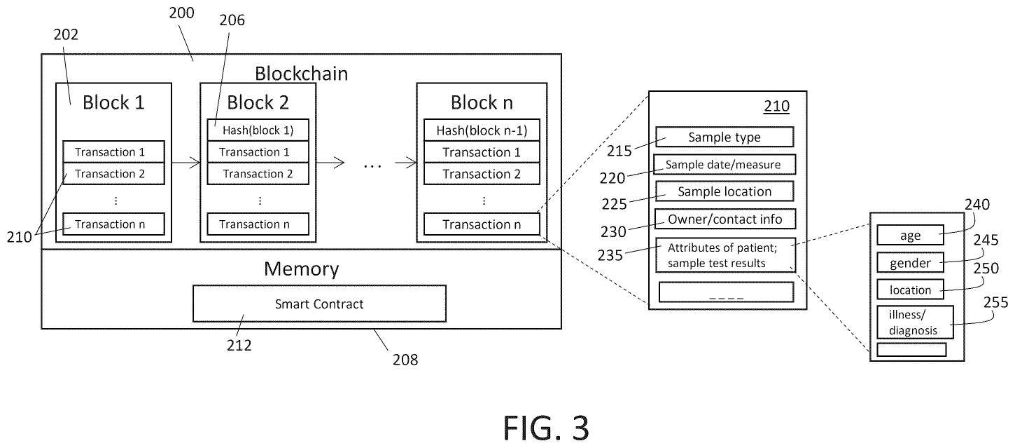

As illustrated with reference to FIG. 3, a blockchain 200 stored at a node 110 includes a plurality of blocks 202. Each block 202 is a data structure that includes data representing transactions 210 that are submitted by users for purposes of storing and accessing information. In the context of the present disclosure, such information stored on the blockchain are generated as transactions regarding patients' biospecimens or biological samples and storage locations and other related information about the stored biospecimens/samples.

As referred to herein, a “biospecimen”, “specimen”, patient sample or “sample” are used interchangeably and refer to a type of physical biological fluid e.g., blood, serum, sputum, saliva, or biological tissue, e.g., skin, muscle, bone, that is taken from a patient for clinical (e.g., diagnostic/treatment) and/or non-clinical purposes. The provision of a sample may be provided within the construct of a health care service provider network (e.g., Enzo laboratories) or external 3^{rd} party laboratories or out-of-network service providers. An associated fee can be assessed to the external or out-of-network third party laboratory for the use of the blockchain for storing the location information of the stored sample and/or related information on the blockchain.

As referred to herein, a “clinical” use of a sample and/or related information refers to the use of the sample/related information, e.g., by a doctor or like professional, for diagnostic and/or treatment purposes of a patient.

As referred to herein, “non-clinical” use of a sample and/or information related to the sample refers to the use of the sample/related information for non-diagnostic purposes, e.g., for subsequent use by a third party, e.g., a researcher at a university, for purposes of research, e.g., statistical analysis. An associated fee can be assessed to the third party for the subsequent use of the blockchain to obtain such a sample and/or information relating to stored samples by the third party.

Referring to FIG. 3, a submitted transaction 210 can include plural searchable data fields relating to the storage of a patient's biospecimen or sample and associated information about the stored biospecimen or sample including, but not limited to: a sample type data field 215 indicating a type of physical biological fluid e.g., blood, serum, sputum, saliva, or biological tissue, e.g., skin, muscle, bone, that is taken from a patient; a sample measure data field 220 indicating a date the sample was taken from the patient (or donated) and a measure of the sample taken, e.g., a weight unit, a mass unit, and/or volume unit value; a sample location data field 225 indicating a physical location where the biospecimen sample is stored, the date the sample was or is to be stored, and may include attributes of storage details (e.g., storage container, storage temperature). The sample storage location can be an address of a physical location, e.g., a laboratory the receives samples and conducts analysis of samples, a hospital, third party specimen storage bank, a university, a doctor's office, etc. and/or a code (e.g., a laboratory code). A further data field 230 in the transaction 210 can indicate a current owner of the sample, e.g., a university, a doctor's office, a hospital, a healthcare entity, a laboratory that receives samples and conducts analysis of samples, a third party specimen storage facility (e.g., a blood bank), and/or include a current address and/or name of a contact or like entity authorized to provide the sample for clinical/non-clinical (e.g., research) use. A further data field 235 in the transaction 210 can indicate any of the attributes of the patient from who the sample was supplied.

In an embodiment, the attributes field 235 of the patient providing the sample can include information such as a filtered, sub-set of medical information about the subject taken from the patient's electronic medical record (EMR) that is generated and used by healthcare professionals. Such medical information may include patient demographic information stored in accordance with the HL7 messaging standard (e.g., HL7 Version 3) for sharing electronic health information used in the clinical practice, the management, delivery, and evaluation of health services (See HL7.org). As shown in FIG. 3, such patient attribute data fields can include, but is not limited to: a field 240 indicating an age of the patient, a field 245 indicating the patient's gender, a field 250 indicating the patient's general geographic location, e.g., county, city, state, etc.; and a field 255 indicating that patient's particular illness/diagnosis that necessitated the need to take the sample and/or a test results of the patient's sample. The transaction 210 can further include a data such as a sample identifier or specimen identifier (not shown) for linking the transaction to the associated sample's storage location and/or contact.

As mentioned, in embodiments, for blockchain immutability, any block added to the blockchain is cryptographically linked to prior blocks using the hash function (e.g., SHA-1 (Secure Hash Algorithm 1). As new transactions 210 are submitted to the blockchain 200 and validated, additional blocks 202 are generated and appended to the blockchain 200. Each new block 202 includes a hash 206 of the immediately previous block 202. For example, block 2 includes a hash of block 1, block n includes a hash of block n-1, etc.

In an embodiment, in association with the blockchain, is a framework for the execution of smart contracts. In an embodiment, the smart contract is a program stored on the blockchain platform and can run when predefined conditions are fulfilled. As shown in FIG. 3, at a memory 208 associated with the blockchain node is a program module 212 (a smart contract) including program elements which run at the local blockchain node. Smart contracts can include a self-executing agreement between parties that have all of the relevant covenants spelled out in code, and settle automatically, e.g., depending on fulfilment of certain conditions or trigger events. In an embodiment, smart contract 212 can be used in a fee/billing arrangement, e.g., between the blockchain owner and: 1) entities that store patient samples and submit related information to the blockchain about the stored sample that can be subsequently accessed/used for clinical or non-clinical purposes; or 2) entities such as users/researchers that search the blockchain to obtain information about the stored samples and associated sample information and/or obtain the physical sample from the owner of the sample. The smart contract can include program logic, source code, scripting language or compiled machine code and can comprise conditions in terms of program logic, and consequences in terms of program logic, wherein the consequences are activated or executed if certain conditions are fulfilled.

By leveraging blockchain technology, a smart contract can perform tasks that are written into the smart contract code including, a billing task (billing an entity for storing a sample on the blockchain, or billing an entity for using a sample or related information stored on the blockchain), or perform a notification task, e.g., for notifying a sample owner about a particular event concerning use of their sample. In an embodiment, the smart contract on the blockchain will bill per transaction and notify sample owner via the web application or mobile device application. Such task can include a transaction between the parties and the blockchain can be updated once the transaction process is completed. The smart contract cannot be revoked, denied, or reversed, since decentralized execution removes them from the control of any one party.

In the context of blockchain-based biological sample storage, smart contracts can include state dependent terms that may be based on events that occur in the future. For example, a smart contract may include a term that the owner of a submitted sample receives a fee for the submission of a sample on the blockchain and/or subsequent use/access of that owner's sample and/or a term that a fee be assessed to a client for performing a search and generating a search result that includes sample information accessed from the blockchain. At the time that the smart contract is validated and appended to the blockchain, the future use of the sample or related sample information is not known. The receipt of a fee for a use of the blockchain sample/information may be considered a state dependent term. In an embodiment, physical samples will be removed from the blockchain or otherwise are non-existent to the blockchain. However, any de-identified data gathered from the sample will be stored indefinitely.

For example, in an embodiment, multiple nodes store a distributed or shared document (ledger) that holds the transactions data including the location of the sample, e.g., indicated as a code or a textual content located in a dedicated transaction text field. Additional information provided in the transaction and stored on a blockchain node include anonymized (cleansed) patient-related data, e.g., patient age, gender, area, or geographic location and/or illness or diagnosis. For an assessed fee, the blockchain system can issue a certificate of authority to clients wishing to gain access to the blockchain, e.g., for purposes of querying the blockchain for information relating to samples or for location information to obtain the physical sample for any clinical or non-clinical (e.g., research) purpose. Certificates of Authority (CAs), in an aspect, can be issued when all the nodes in the network run a blockchain consensus algorithm to verify that the client has access to the network. Such consensus can include Proof of Stake (POS), which is a known mechanism utilizing randomly selected miners to validate transactions; or Proof of Work which is a known mechanism using a competitive validation method to confirm transactions and add new blocks.

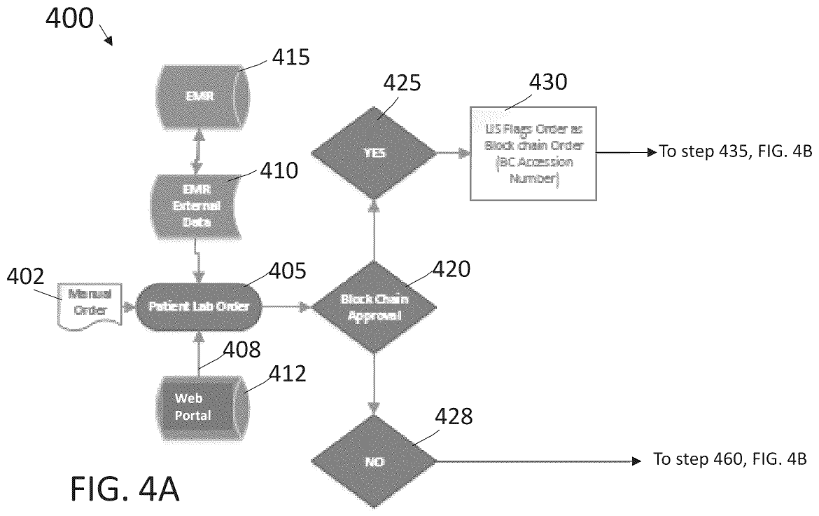

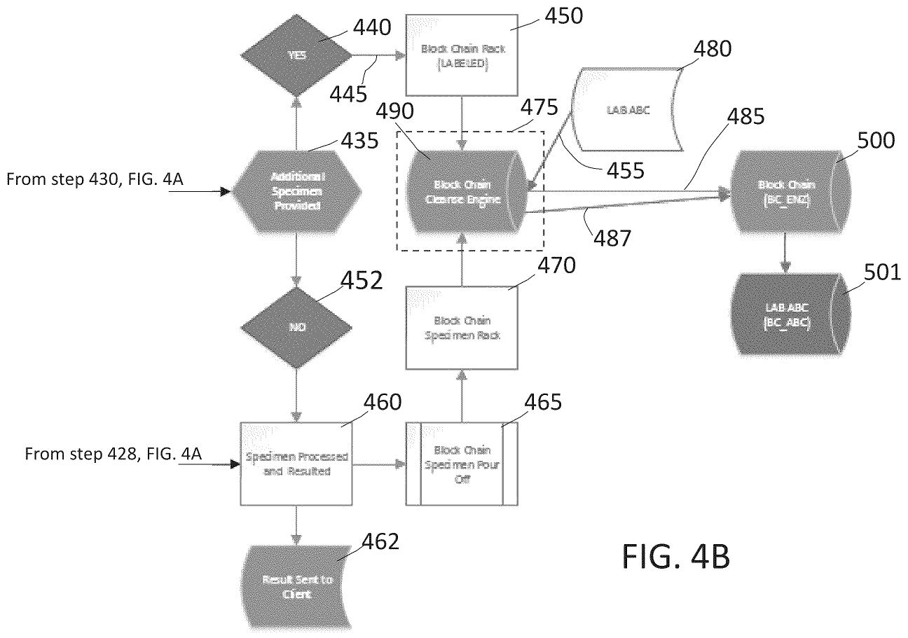

In an embodiment use of the biological sample storage blockchain network to remotely store biological samples or specimens and related information about the stored sample on the computing network 100 of FIG. 1, reference is now had to FIG. 4A which shows an overall method 400 of a sample storage workflow using the blockchain. Initial step 405 refers to the step of taking of the patient's biospecimen/sample such as blood, urine, sputum, tissue, etc. at a doctor's office, hospital, or other appropriate medical facility. The taking of the patient's sample can be in response to a doctor's prescription, e.g., a received doctor's manual order 402, or in response to a received electronic medical record (EMR) 415, or in response to a request received from a user (e.g., a phlebotomist) through a web-based portal 412. In an embodiment, at 405, the patient's sample is taken at a clinical lab for analysis. In a further embodiment, a previous biospecimen or previous sample taken at the doctor's office or hospital (or like medical facility) can be conveyed to the clinical lab for analysis along with an electronic medical record (EMR). In an embodiment, besides manually taking a patient sample at the laboratory, a patient's lab order is sent to the lab from an external doctor's office/hospital/medical facility for processing by an electronic medical record/electronic health record (EHR), e.g., along with collected historical data 410 on a patient upfront from EMR 415. Such a medical record can include a consent of the patient authorizing the taking, storing, and using of the sample. In a further embodiment, a patient can authorize the taking of an additional biospecimen or excess sample for further related storage/clinical use by the patient's doctor and/or stored for non-related use, e.g., such as for purposes of research by third parties. In either use-case, the user has to provide such consent for such storage and further related or non-related use of the sample. The patient's consent/authorization to take a sample or additional samples can be provided by the patient at the laboratory taking the sample and recorded in the patient's medical record. Alternatively, the medical record can record the patient's consent and be transmitted to the lab with the sample to be analyzed. In an embodiment, a patient's consent 408 for storage/usage of their sample or consent for the laboratory to take/store any additional or excess biospecimen or sample for later use (diagnosis or treatment) or for later non-clinical use can be provided to the user of the web-based portal 412 which portal allows a user (e.g., a phlebotomist) at a patient service center or a staff member at a client location to order the taking of patients' samples.

Once the patient's lab order is processed at the lab, e.g., including the taking of a sample or biospecimen from the patient at 405, a decision is made at 420 as to whether the patient's sample is to be stored as a transaction on the sample storage blockchain. Such a decision is responsive to whether the sample meets a threshold criterion, such as amount of sample, e.g., the patient's biospecimen meeting a weight, mass, or volume threshold. Such a decision further hinges on the obtaining of the patient's consent authorizing the storage of the patient's sample (i.e., whether it be excess biospecimen (e.g., “specimen pour off”) remaining from the sample after clinical use/diagnosis by the lab, or whether it be additional specimen taken from the patient at the lab). Thus, in an embodiment, any future non-clinical use of stored biological samples is communicated transparent to the actual donors, e.g., a patient or sample donor executes informed consent forms that authorize and enable storage and future research use of their biological specimens. These patient/donors are typically informed about what personal or medical information (e.g., age, gender) about them will be used in the research, who it might be shared with, and what safeguards are in place to protect their confidentiality.

Upon obtaining such patient authorization, and responsive to ensuring or certifying that a sample weight or volume threshold criteria is met for the stored biological sample added on the blockchain, approval workflow process continues at the yes decision indicated at 425 for blockchain processing. At this point, at 430, the sample order is flagged as a blockchain order by laboratory information systems (LIS), given a Blockchain ID (BCID) and a BC Accession number which is a unique alphanumeric identifier that is given to blockchain results that allows seamless data sharing across the healthcare ecosystems. Processing then continues at step 435, FIG. 4B. Otherwise, at 420, if no approval is provided for sample storage on the blockchain, the denial workflow is followed and processing continues at the no decision indicated at 428 in which case processing continues at step 460, FIG. 4B.

Referring to FIG. 4B, continuing from step 430, FIG. 4A, the blockchain approval workflow process continues to 435 which depicts the method step of processing the authorized additional specimen taken from the patient by the analysis laboratory for blockchain storage in accordance with the patient authorized clinical and/or non-clinical use. The processing of the additional patient specimen is initiated when the additional specimen is provided as indicated at the “yes” block 440. Then, at 445, FIG. 4B, the additional specimen, e.g., a vial(s), is (are) sent to a blockchain rack 450 for appropriate collection and labeling with blockchain ID number and any other patient related information. In embodiments, the function of the blockchain rack 450 is to identify the specimen as a Blockchain specimen and separate it from day-to-day specimens, e.g., any specimens not destined for storage on the blockchain. At the blockchain rack 450, there can be generated a transaction related to the received specimen for addition to a block that is to be stored on the blockchain. Such information provided in a transaction includes the location of the physical biospecimen or sample, i.e., the location where the sample is physically stored. This sample storage location may be indicated as a code(s) or as text populated in a suitable message field(s). The information can further include any related information about the sample, e.g., sample type, a sample measure such as a weight, volume, storage container attributes, etc., and any related and filtered information about the patient supplying the sample, e.g., patient age, gender, geographic location, diagnosis of an illness, race, ethnicity, smoker vs. non-smoker, etc. In embodiments, the medical record of the patient supplying the additional specimen follows the patient's additional specimen and the patient's medical record including the related sample storage information is communicated to a blockchain processing server or blockchain node 475 for cleansing (filtering) by a blockchain cleansing engine 490 for research purposes.

Particularly, blockchain cleansing engine 490 performs processes and routines for sanitizing and de-identifying of any patient identity information from the received medical record. In an embodiment, the cleanse engine is an analytic process built in an object-oriented database. While the cleanse engine can employ a bi-directional data transport interface between EMR/EHR vendors and the blockchain owner, in an embodiment, the cleanse engine will not directly communicate with EMR/EHR systems; rather, an application program interface (API) runs between an existing interface engine and the cleanse engine for enabling transfer of data between these entities. From received EMR/EHR data, blockchain cleansing engine 490 can parse/remove data such as the patient's name, residence address, phone number, social security number, and any like patient identifying information while retaining general demographic information about the sample such as patient age, gender, a diagnosis, and area or geographic location of the patient. Once the transaction information is cleansed, the transaction or block of transactions 485 can be processed by blockchain processing server or node 475 and placed onto the blockchain 500 for storage thereon. In embodiments, patient data is parsed/cleansed within the cleanse engine 490 by utilizing blockchain identifiers within the HL7 message.

In an additional embodiment, a 3^{rd} party or “out-of-network” laboratory 480 can take and store patient samples and related sample storage location information on the blockchain. In such embodiments, the information and/or stored samples can be subsequently used for clinical and/or non-clinical purposes. Via a smart contract(s) executing on the blockchain, such a use event can trigger a fee assessed to the third party for the use of the blockchain for storing and accessing samples. In this embodiment, the samples can be taken at and/or provided by a 3^{rd} party laboratory 480 (e.g., Lab “ABC”) who can send patient healthcare information including the storage location of the samples taken from the patient to the blockchain processing server or node 475. Via the blockchain processing server node 475, an interface can be provided through which data can be collected including the sample storage location and related sample information for storage on the blockchain. In this embodiment, at the 3^{rd} party laboratory 480, the patient lab order is processed through the LIS, the lab analysis results are released in a message 455 formatted in accordance with the HL7 data transfer messaging standard. The HL7 results message 455 that is released will include all the standard patient identification information and is paired to a Blockchain ID (obtained at step 430). Additional fields in the HL7 or a JavaScript Object Notation (JSON) message 455 will contain the location of the physical biospecimen or sample, i.e., the location where the sample is physically stored, and related information about the sample, e.g., sample type, weight, mass, volume, storage container attributes, etc. This sample storage location may be indicated as a code(s) or as text populated in a suitable HL7 (or JSON) message field(s). The HL7 (or JSON) message providing this sample information and the patient's lab analysis results is electronically sent by the laboratory 480 for receipt at a blockchain processing server or node 475 where it is then passed for cleansing by blockchain “cleanse engine” 490. Particularly, blockchain cleansing engine 490 performs processes and routines for sanitizing and de-identifying, i.e., anonymizing by removing any patient identity information from the received medical record, e.g., HL7 (or JSON) message 455, related to the sample. The storage location of the sample by the laboratory 480 and the remaining cleansed data about the patient is then packaged as a transaction at the blockchain processing server or node 475 and added to a block 487 that is stored on the blockchain 500. A user, via a client application run on a computer or through a web-based portal, or via any preferred method of delivery, can access the analysis result. The 3^{rd} party laboratory 480 is further authorized to access the blockchain at 501.

Referring back to 435, FIG. 4B, in the case that the patient did not authorize or consent to the taking of any additional biospecimen (beyond the amount of the ordered sample taken for immediate diagnostic/treatment purposes), but approval has been provided for storage on the blockchain, the denial workflow processing path is taken at the “no” block 452 where the system waits at 460 for the laboratory results of the patient's specimen. During processing, the order is given a Blockchain ID, and the ID is then sent to the blockchain cleanse engine 490 for later use, as described below.

At 460, once the originally obtained specimen has been processed and laboratory analysis results reported, the process continues to 465 where any excess specimen remaining from the original sample is “poured off” (e.g., packaged for further storage/use) and placed on the block chain specimen rack at 470. It is verified that the measured amount of specimen “pour off” meets the criteria for storage and subsequent clinical or non-clinical use commensurate with the patient consent. Subsequently, the process continues and the “poured off” sample portion and related storage location information is provided to the blockchain processing server or blockchain node 475 where the blockchain cleanse engine sanitizes, de-identifies or otherwise “anonymize” any patient identity information for research purposes, particularly by removing such information from the medical record related to the poured off patient sample before storage on the blockchain 500. In the sanitizing process at the cleanse engine, personal identification information forms the patient's medical record is removed (e.g., patient name, address, phone number, social security number, etc.). Any patient information that may be retained with the sample after being cleansed includes the patient age, gender, race, ethnicity, diagnosis, whether the patient was a smoker or non-smoker and area or geographic location of the patient. In parallel to the specimen pour off and blockchain cleanse engine processing, at 462, the lab analysis result is rendered available to the client, e.g., via the client's preferred method of delivery, application, or web-based portal.

Further, continuing from step 428, FIG. 4A indicating no blockchain approval, a blockchain disapproval workflow path runs process steps 460, 465, 470 of FIG. 4B. However, in such a case, the sample is not stored at the blockchain 500. Rather, all cleansed and sanitized results sample processing results and related sample location storage information for the patient's sample is stored at the cleanse engine as historical data.

That is, if no additional specimen is received from the patient, the no path is followed at 452, and the diagnostic analysis/testing results of the original ordered laboratory specimen are obtained at 460 and this laboratory analysis result is sent to a client application at 462 via the client's preferred method of delivery. At 460, once the specimen has been processed and results received, the process continues to 465 where the specimen “pour off”, i.e., excess specimen remaining from the original sample, is obtained and placed on the block chain specimen rack at 470.

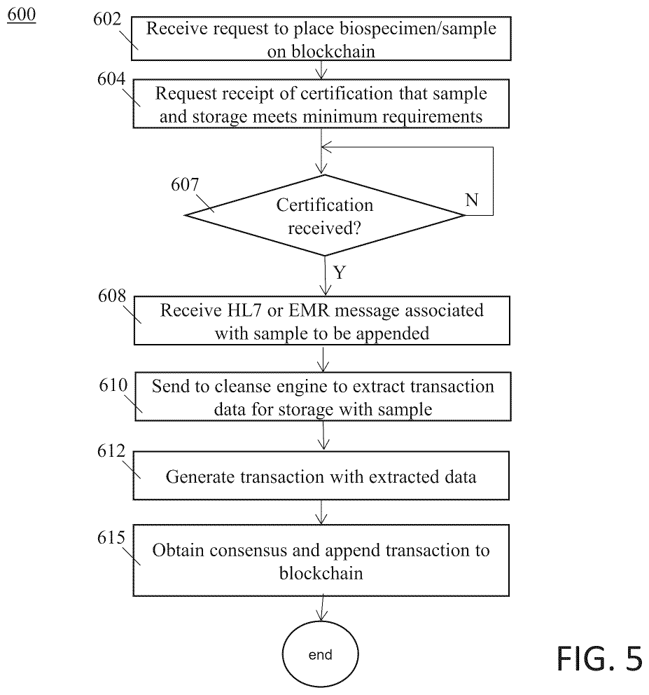

In a further aspect, FIG. 5 shows a method 600 run by a smart contract executing on a blockchain node to enable a user to add a biological sample, its storage location, and related sample information. While the method 600 is described for the sake of convenience and not with an intent of limiting the disclosure as comprising a series and/or a number of steps, it is to be understood that the process does not need to be performed as a series of steps and/or the steps do not need to be performed in the order shown and described with respect to FIG. 5, but the process may be integrated and/or one or more steps may be performed together, simultaneously, or the steps may be performed in the order disclosed or in an alternate order.

In FIG. 5, a first step 602 represents the receipt from a submitter, e.g., an owner or possessor of a patient's biological sample, a request to place the patient's sample and related sample storage location on the blockchain. At 604, if not already authorized, the blockchain smart contract may issue and communicate to the submitter a request for certification that the sample and/or its storage facility meets minimum requirements, e.g., the sample meets a minimum measure amount (weight, mass, or volume) or the physical sample storage facility, storage location or environment meets certain requirements, and/or any attributes of the physical sample storage container meets any threshold physical requirements. At 607, FIG. 5, the system idles until it is determined that a certification or a verified proof has been received that the sample and/or its storage facility/container meets minimum requirements. Once certification or token is received at 607, the process proceeds to 608 which represents the further receipt of an HL7 message, or an electronic medical record (EMR) associated with the patient whose sample is being stored. Step 608 can include the pairing of the received sample and patient's HL7 or like EMR to an assigned Blockchain ID number. At 610, upon pairing the HL7 message with the Blockchain ID, the HL7 or EMR is sent to a blockchain cleanse engine to sanitize and de-identify the patient information contained therein. In an embodiment, the cleanse engine can look for predetermined message fields in the HL7 or EMR structure and only extract certain content from those pre-determined fields. Such patient information extracted can include, but is not limited to: the patient's age, gender, race, ethnicity, patient's geographic location, the patient's diagnosis, or illness, etc. In an alternative embodiment, the cleanse engine can look for predetermined message fields in the HL7 JSON or EMR structure and remove certain content from pre-determined fields. Such patient information removed from the pre-determine fields includes, but is not limited to: the patient's name, the patient's residence address, the patient's social security number, the patient's physical attributes, etc. Continuing to 612, FIG. 5, there is depicted the further steps of generating a transaction for storage on the blockchain. The generated transaction will include pre-determined fields including the patient information extracted from (or remaining in) the patient's associated HL7 message or EMR and include the sample type and sample storage location. Finally, at 615, the blockchain network of peer nodes are invoked to obtain a consensus which, when received, result in appending the generated transaction to the blockchain 500.

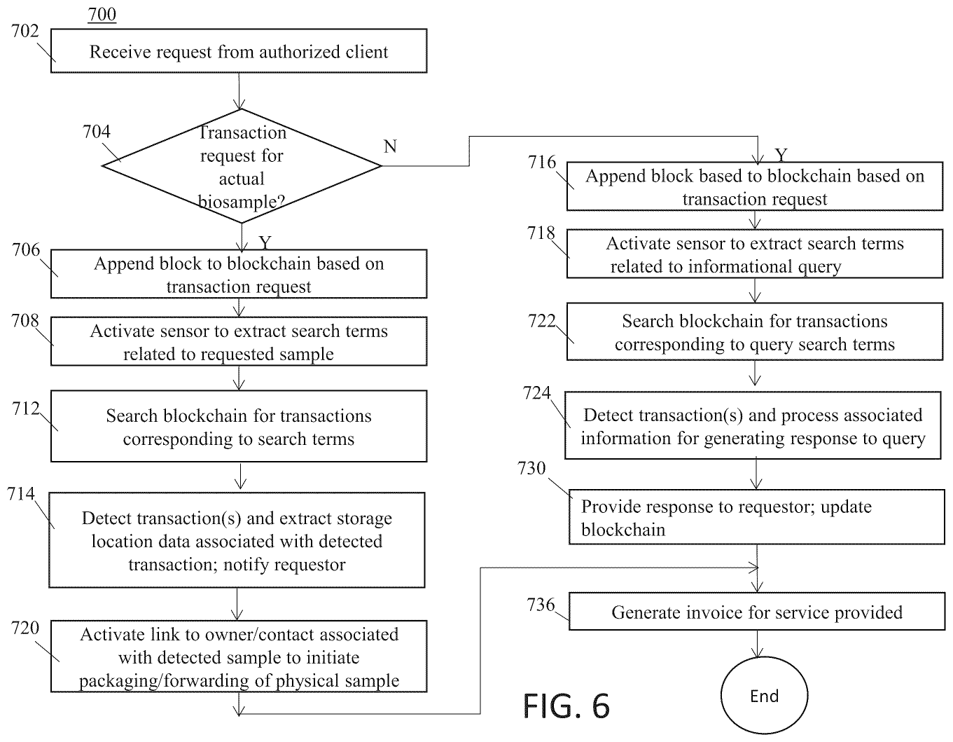

In a further aspect, FIG. 6 shows a method 700 run by a smart contract executing on a blockchain node to provide access (e.g., remote access) to the blockchain for an exemplary sample use. While the method 700 is described for the sake of convenience and not with an intent of limiting the disclosure as comprising a series and/or a number of steps, it is to be understood that the process does not need to be performed as a series of steps and/or the steps do not need to be performed in the order shown and described with respect to FIG. 6, but the process may be integrated and/or one or more steps may be performed together, simultaneously, or the steps may be performed in the order disclosed or in an alternate order.

In FIG. 6, a first step 702 represents the receipt from a requestor, e.g., a doctor or researcher, desirous of obtaining a patient's biological sample(s) for clinical or non-clinical use, or alternatively obtain certain anonymized information related to a patient(s) stored biological sample(s). Such receipt of a remote user request can initiate the generation of an associated request transaction to be appended to the blockchain. At step 704, a determination is made as to whether the transaction request is for the actual physical biosample or, alternatively, part of a general query concerning a request to obtain certain information about patient(s) stored biological sample(s), e.g., for non-clinical use. For example, a researcher may be enabled through the client application or web-based portal to submit a query pertaining to a patient(s) sample(s). An exemplary user query can include, for example, a request for information about physical samples from a number of patients of a certain age group or gender having a certain illness in a certain geographic location. In an embodiment, a query may include a criteria for searching including one or a combination of: patient(s) age, gender, geographic location, diagnosis of an illness, race and ethnicity, smoker vs. non-smoker, etc.

If it is determined that the received request is for an actual physical stored sample, the process proceeds to 706 where a transaction or block is appended to the blockchain based on the sample request. Then, at 708, a ledger sensor is activated to extract search terms of predefined fields having search words that can be used to conduct a search on the blockchain for obtaining the requested sample or information pertaining to stored samples. Such search terms extracted can include such search words including, but not limited to a combination of one or more of: the type of sample being requested, an age of the patient supplying the sample, whether the patient was a smoker or non-smoker, a date of sample collection, a geographic location of where the patient resided, or a type of illness or diagnosis associated with the patient whose sample was taken and stored. Continuing to 712, the blockchain node implements smart contract functionality to initiate a search of the blockchain for transaction(s) associated with stored patients' sample(s) meeting the criteria of the extracted search terms (e.g., sample type, patient age, diagnosis, smoker vs. non-smoker, geographic location). Then, at 714, upon detecting transactions on the blockchain representing stored samples that meet the desired search criteria, the smart contract performs extracting storage location data associated with samples in detected transaction. At this point, the requestor can be notified of the particular sample storage location and related patient information that has been anonymized. If the requestor approves of the sample(s), the process proceeds to 720 where the smart contract automatically activates a link to the storage location owner/contact associated with the detected sample(s) to reserve that sample for the requestor's use and/or initiate a packaging/forwarding or transfer of the physical sample(s) to the requestor according to pre-defined terms. Whether the physical biological sample is first reserved for packaging and conveyance to the requestor, or whether the physical biological sample is first automatically packaged and sent to the requestor, an associated transaction is formed to reflect the reservation of the sample and/or the sample(s) conveyance in satisfaction of the received request. Further, upon reserving and/or initiating transfer of the sample, the process proceeds to 736, FIG. 6 where the smart contract can initiate generation of an invoice transaction for the service in connection with the requestor's sample request and fulfilment, or otherwise, debit a pre-determined monetary or cryptocurrency account, e.g., Bitcoin (BTC), Ethereum (ETH) or a custom cryptocurrency account, associated with that requestor.

Alternatively, returning to step 704, FIG. 6, if it is determined that transaction requestor is an authorized researcher requesting information relating to stored samples, the process proceeds to 716 where a transaction or block is appended to the blockchain based on this informational request. Such an example information request can be: the number of samples that can be located for patients of one or a combination of attributes including but not limited to: a certain age group, gender, a particular geographic area, and with a particular diagnosis or illness, race or ethnicity, smoker vs. non-smoker, etc. Then, at 718, a ledger sensor is activated to extract search terms of predefined fields relating to the informational query received. Such search terms extracted can include a combination of one or more of: information relating to a particular type of sample being requested for patients of a certain age, a geographic location of where those patients resided, or a type of illness or diagnosis associated with the patients whose samples were stored. Continuing to 722, FIG. 6, the smart contract extracts search terms such as the age, the geographic location, and the diagnosis, and initiates a search on the blockchain for transactions associated with samples stored on the blockchain having associated information that meets such search criteria. Any relevant transactions about samples stored on the blockchain that meet search criteria can be linked to the query for use in generating a response message. Then, continuing to 724, the executing smart contract detects one or multiple linked transaction(s) meeting the associated search criteria and processes the transaction information for generating a response to the query. This may entail collecting information from the plurality of linked transaction or tallying a number of the plurality of linked transactions associated with plural patients' samples that meet the search criteria. For example, a response can include a number of patients found of a certain age group having a certain diagnosis of the certain geographic location. At 730, FIG. 6, this requested response information can be returned to the requestor via the client application or portal front-end in response to query and an associated transaction memorializing the response can be appended to the blockchain. Then, the process proceeds to step 736, FIG. 6 where the smart contract can initiate generation of an invoice transaction for the service in connection with answering the requestor's informational request, or otherwise, debit a pre-determined monetary or cryptocurrency account associated with that requestor.

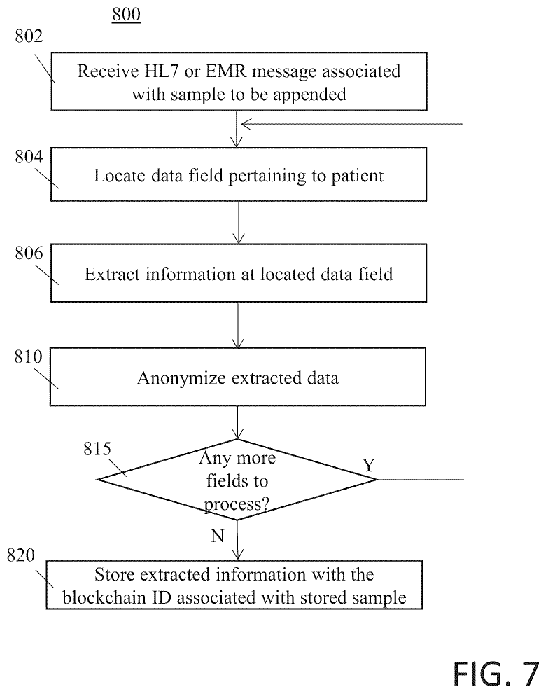

FIG. 7 depicts a method 800 implemented at a blockchain node for cleansing information (at the cleanse Engine 490) related to a patient whose sample is being stored. While the method 800 is described for the sake of convenience and not with an intent of limiting the disclosure as comprising a series and/or a number of steps, it is to be understood that the process does not need to be performed as a series of steps and/or the steps do not need to be performed in the order shown and described with respect to FIG. 7, but the process may be integrated and/or one or more steps may be performed together, simultaneously, or the steps may be performed in the order disclosed or in an alternate order.

In FIG. 7, step 802 depicts the steps of receiving, at the cleanse engine of a blockchain node, a HL7 message or like electronic medical record associated with the patient whose sample is requested to be stored on the blockchain using an assigned blockchain ID number. In an embodiment, the cleanse engine can capture data from HL7 transactions from an internal Laboratory Information System (LIS) or a third party LIS, sanitize/de-identify the data to be sent to the blockchain, and after the data has been cleansed, it is then sent to the blockchain with a blockchain identifier (BCID). In an embodiment, at 804, search processes are run at a cleanse engine at a blockchain node for locating certain data fields in the HL7 message having information that can be used to identify the patient and, at 806, removing (sanitizing or de-identifying) such information. Initially, a location in the HL7 message would be used to provide the blockchain identifier, i.e.—OBR-3 or OBR-10 in the HL7 message. The blockchain identifier (BCID) would be used to determine if the message should go to the blockchain. If the value is NULL, then it would not be sent to the blockchain. For example, such data fields having patient identity information to be removed can be those fields indicating the patient's name, patient residence address, patient social security number or license number, a patient's physical attributes such as height, weight, eye/hair color, the patient's vocation, etc. Continuing at 810, in some instances, rather than wholesale removing the information, rather the data can be anonymized, for example, a patient's age may be replaced with an age range, e.g., 50 years to 60 years, or a patient's address can be anonymized to indicate only a geographic region, e.g., a county, rather than the particular town or municipality within which the patient resides, smoker vs. non-smoker or any other information that is relevant, such as gender, race, ethnicity, and the like. Continuing to 815, a determination is made as to whether any further data fields within the HL7 message remains to be processed for sanitizing or de-identifying. If there are additional fields to process, the process returns back to 804 to repeat the steps 804, 806 and/or anonymize data at step 810. If at 815, it is determined that there are no more fields to process to cleanse the sensitive patient information, the process proceeds to 820 where the remaining anonymized patient data, e.g., age, gender, diagnosis or illness or location, and the associated details about the actual sample, is formed into a transaction along with the Blockchain ID, for storage at the blockchain 500.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM)[SDRAM], a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowcharts and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Moreover, a system according to various embodiments may include a processor, functional units of a processor, or computer implemented system, and logic integrated with and/or executable by the system, processor, or functional units, the logic being configured to perform one or more of the process steps cited herein. What is meant by integrated with is that in an embodiment the functional unit or processor has logic embedded therewith as hardware logic, such as an application specific integrated circuit (ASIC), a field programmable gate array (FPGA), etc. By executable by the functional unit or processor, what is meant is that the logic in an embodiment is hardware logic; software logic such as firmware, part of an operating system, part of an application program; etc., or some combination of hardware or software logic that is accessible by the functional unit or processor and configured to cause the functional unit or processor to perform some functionality upon execution by the functional unit or processor. Software logic may be stored on local and/or remote memory of any memory type, as known in the art. Any processor known in the art may be used, such as a software processor module and/or a hardware processor such as an ASIC, a FPGA, a central processing unit (CPU), an integrated circuit (IC), a graphics processing unit (GPU), etc.

It will be clear that the various features of the foregoing systems and/or methodologies may be combined in any way, creating a plurality of combinations from the descriptions presented above. If will be further appreciated that embodiments of the present invention may be provided in the form of a service deployed on behalf of a customer to offer a service on demand.

The terminology used herein is for the purpose of describing particular embodiments only and is not intended to be limiting of the invention. Unless otherwise specifically defined herein, all terms are to be given their broadest possible interpretation including meanings implied from the specification as well as meanings understood by those skilled in the art and/or as defined in dictionaries, treatises, etc. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. The corresponding structures, materials, acts, and equivalents of all elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment and terminology were chosen and described in order to best explain the principles of the invention and the practical application, and to enable others of ordinary skill in the art to understand the invention for various embodiments with various modifications as are suited to the particular use contemplated.

What is claimed is:

1. A computer-implemented method of managing biological samples using a network of computing nodes, the method comprising:
receiving, at a processor of a computing node, a message request to store information relating to a biological sample taken from a subject;
extracting from the message request, using said processor, location information associated with the subject's stored physical biological sample;
obtaining, using the processor, data associated with a subject providing the stored sample;
appending, using the processor, a transaction to a blockchain stored on one or more computing nodes of said network, said transaction comprising information about the subject's biological sample stored, the associated location information of the subject's stored sample and the data associated with the subject;
receiving, using said processor, queries pertaining to stored biological samples; and
responding to said queries, using the processor, by accessing the information within one or more transactions appended to said blockchain.

2. The computer-implemented method of claim 1, wherein obtaining data associated with the subject comprises:
receiving, by the processor, an electronic medical record having information relating to an identity of the subject providing the stored sample; and
filtering, using the processor, the electronic medical record to extract patient information that does not reveal said subject identity.

3. The computer-implemented method of claim 2, wherein the filtering comprises one or more of: removing subject identity information from predetermined data fields of said medical record or rendering as anonymous subject identity information in said predetermined data fields of said medical record.

4. The computer-implemented method of claim 1, wherein responding to said queries comprises:
obtaining, using said processor, search words located at respective predetermined fields of a query;
linking, by said processor, one or more said transactions appended on said blockchain to said query based on said obtained search words.

5. The computer-implemented method of claim 4, further comprising:
providing a query response message, using the processor, for receipt at a device associated with a requestor, said query response message including information based on said linked one or more transactions.

6. The computer-implemented method of claim 1, wherein a received query relating to a biological sample comprises a request from a requestor to obtain a stored physical biological sample, said method further comprising:
locating on the blockchain, using the processor, a transaction associated with the requested stored physical biological sample;
accessing, using the processor, from the located transaction associated with the requested stored physical biological sample, an information of a contact entity responsible for providing said stored biological sample; and
automatically sending, using the processor, a communication to the contact entity to initiate a packaging of the stored sample requested in the received request, said packaging suitable for conveyance of the biological sample to the requestor.

7. The computer-implemented method of claim 6, further comprising:
sending, using the processor, a communication to the contact entity to reserve, for subsequent conveyance to the requestor, the physical biological sample for use by the requestor.

8. The computer-implemented method of claim 4, wherein said responding to said queries by accessing the information within one or more transactions comprises:
collecting, using the processor, information from each the one or more linked transactions pertaining to the obtained search words; and
generating, using the processor, a response to the query message using the collected information.

9. The computer-implemented method of claim 8, wherein a further query message request for information comprises: a request for one or more available biological samples from subjects meeting a predetermined criterion.

10. The computer-implemented method of claim 9, wherein said collecting, using the processor, information from each of the one or more transactions pertaining to the requested information further comprises: identifying one or more transactions associated with subjects meeting the predetermined criteria.

11. The computer-implemented method of claim 10 wherein said predetermined criteria comprises: a biological sample type from subjects of a particular age group in a certain geographic location.

12. A non-transitory computer readable medium comprising instructions that, when executed by at least one hardware processor, configure the at least one hardware processor to:
receive a message request to store information relating to a biological sample taken from a subject;
extract, from the message request, location information associated with the subject's stored physical biological sample;
obtain data associated with a subject providing the stored sample;
append a transaction to a blockchain stored on one or more computing nodes of said network, said transaction comprising information about the subject's biological sample stored, the associated location information of the subject's stored sample and the data associated with the subject;
receive queries pertaining to stored biological samples; and
respond to said queries by accessing the information within one or more transactions appended to said blockchain.

13. The non-transitory computer readable medium according to claim 12, wherein to obtain data associated with the subject, the instructions further configure the at least one hardware processor to:
receive an electronic medical record having information relating to an identity of the subject providing the stored sample; and
filter the electronic medical record to extract patient information that does not reveal said subject identity.

14. The non-transitory computer readable medium according to claim 13, wherein to filter the electronic medical record, the instructions further configure the at least one hardware processor to:
one or more of: remove subject identity information from predetermined data fields of said medical record or render as anonymous subject identity information in said predetermined data fields of said medical record.

15. The non-transitory computer readable medium according to claim 12, wherein to respond to said queries, the instructions further configure the at least one hardware processor to:
obtain search words located at respective predetermined fields of a query;
link one or more said transactions appended on said blockchain to said query based on said obtained search words.

16. The non-transitory computer readable medium according to claim 15, wherein the instructions further configure the at least one hardware processor to:
provide a query response message for receipt at a device associated with a requestor, said query response message including information based on said linked one or more transactions.

17. The non-transitory computer readable medium according to claim 12, wherein a received query relating to a biological sample comprises a request from a requestor to obtain a stored physical biological sample, the instructions further configuring the at least one hardware processor to:
locate on the blockchain a transaction associated with the requested stored physical biological sample;
access from the located transaction associated with the requested stored physical biological sample, an information of a contact entity responsible for providing said stored biological sample; and
automatically send a communication to the contact entity to initiate a packaging of the stored sample requested in the received request, said packaging suitable for conveyance of the biological sample to the requestor.

18. A computer-implemented system for managing biological samples using a network of computing nodes, the system comprising:
a memory storage device storing program instructions; and
a hardware processor having circuitry and logic to execute said program instructions to configure the network of computing nodes, the hardware processor coupled to said memory storage device and in response to executing said program instructions is configured to:
receive a message request to store information relating to a biological sample taken from a subject;
extract, from the message request, location information associated with the subject's stored physical biological sample;
obtain data associated with a subject providing the stored sample;
append a transaction to a blockchain stored on one or more computing nodes of said network, said transaction comprising information about the subject's biological sample stored, the associated location information of the subject's stored sample and the data associated with the subject;
receive queries pertaining to stored biological samples; and
respond to said queries by accessing the information within one or more transactions appended to said blockchain.

19. The computer-implemented system according to claim 18, wherein to obtain data associated with the subject, the hardware processor is further configured to:
receive an electronic medical record having information relating to an identity of the subject providing the stored sample; and
filter the electronic medical record to extract patient information that does not reveal said subject identity.

20. The computer-implemented system according to claim 19, wherein to filter the electronic medical record, the hardware processor is further configured to:
one or more of: remove subject identity information from predetermined data fields of said medical record or render as anonymous subject identity information in said predetermined data fields of said medical record.

21. The computer-implemented system according to claim 18, wherein to respond to said queries, the hardware processor is further configured to:
obtain search words located at respective predetermined fields of a query;
link one or more said transactions appended on said blockchain to said query based on said obtained search words.

22. The computer-implemented system according to claim 21, wherein the hardware processor is further configured to:
provide a query response message for receipt at a device associated with a requestor, said query response message including information based on said linked one or more transactions.

23. The computer-implemented system according to claim 18, wherein a received query relating to a biological sample comprises a request from a requestor to obtain a stored physical biological sample, wherein the hardware processor is further configured to:
locate on the blockchain a transaction associated with the requested stored physical biological sample;
access from the located transaction associated with the requested stored physical biological sample, an information of a contact entity responsible for providing said stored biological sample; and
automatically send a communication to the contact entity to initiate a packaging of the stored sample requested in the received request, said packaging suitable for conveyance of the biological sample to the requestor.
